(19) United States
(12) Patent Application Publication
(10) Pub. No.: US 2020/0179441 A1
(43) Pub. Date: Jun. 11, 2020

(54) SYNERGISTIC COMPOSITIONS FOR INCREASING MITOCHONDRIAL FUNCTION AND ENERGY

(71) Applicant: H2 Water Technologies Ltd., Vancouver (CA)

(72) Inventors: Alexander TARNAVA, Coquitlam (CA); Richard James HOLLAND, Vancouver (CA)

(21) Appl. No.: 16/608,551

(22) PCT Filed: Apr. 27, 2018
(86) PCT No.: PCT/CA2018/050493

§ 371 Date: Oct. 25, 2019

Related U.S. Application Data
(60) Provisional application No. 62/490,925, filed on Apr. 27, 2017.

(51) Int. Cl. A61K 33/06; A61K 31/437; A61K 9/20; A61K 31/706; A61K 47/26; A61K 31/19; A61K 47/12

(52) U.S. Cl. CPC A61K 33/06; A61K 31/437; A61K 9/20; A61K 47/12; A61K 47/26; A61K 31/19; A61K 31/706

(57) ABSTRACT

The present invention provides a composition or kit that dissolves in water to produce hydrogen rich water for use in increasing mitochondrial function and energy production. The composition or kit contains magnesium metal, i.e., elemental magnesium, an edible acid and either pyrroloquinoline quinone (PQQ) in any of its forms, a NAD+ precursor such as nicotinamide mononucleotide or nicotinamide riboside or a combination thereof. In water, the magnesium metal and edible acid react to produce magnesium ions and H2, which dissolves in the water.

### BACKGROUND OF THE INVENTION

As we age, multiple issues arise in healthy mitochondrial function from impairment of mitochondrial biogenesis, to decreases in levels of the NAD+ coenzyme. Free radical damage inside the mitochondria is proposed as a potential causative factor. If mitochondrial mutations as a result of internal damage through free radicals allow replicated mitochondria to avoid being broken down by our lysosome, energy production will decrease while the rate of mitochondrial damage via free radicals will increase. Issues such as dysregulation of homeostatic function of our redox and inflammatory balances will arise, leading to increases in advanced glycation end product (AGE) crosslinking, which has been shown to suppress NAD+ levels in part by reducing nicotinamide phosphoribosyltransferase leading to SIRT1 depletion. Decreased SIRT1 levels lead to excess activation of NF-κB p65 and increased transcription of inflammatory genes, such as TNF-α, contributing to insulin resistance. As these issues begin to arise, a cyclical cause and effect will occur, as each contributes to the others dysregulation. Simply increasing mitochondrial biogenesis or NAD+ levels without addressing root issues will do little to decrease the deleterious effects of aging.

Accordingly, there is a need for simultaneously protecting against damage while increasing beneficial responses, so that healthy mitochondrial and energy function may be returned to individuals in middle and advanced age.

### SUMMARY OF THE INVENTION

The invention provides compositions, kits, and methods of use for increasing mitochondrial function and energy.

In one aspect, the invention provides a composition, e.g., a tablet, including magnesium metal; at least one acid; and pyrroloquinoline quinone (PQQ) or salt thereof or a NAD+ precursor. In one embodiments, the composition includes both PQQ or salt thereof and the NAD+ precursor. Alternatively, the composition includes only one of PQQ or salt thereof and the NAD+ precursor. The magnesium metal may include flakes, e.g., −325 mesh flakes. The magnesium metal may be of 200 mesh or smaller. In some embodiments, the amount of magnesium metal is 5-500 mg. The at least one acid may be selected from the group consisting of maleic acid, succinic acid, malic acid, fumaric acid, formic acid, citric acid, ascorbic acid, oxalic acid, and tartaric acid, or a mixture thereof. In certain embodiments, the at least one acid is of 60 mesh or smaller. The amount of the at least one acid may be 30-4000 mg.

The composition may further include a binding agent, e.g., mannitol, xylitol, maltose, dextrose, or lactose. In other embodiments, the composition further includes water-soluble lubricant, e.g., sodium stearyl fumarate or steric acid. The amount of PQQ or salt thereof may be 1-300 mg. The amount of NAD+ precursor may be 15-2500 mg. In certain embodiments, the NAD+ precursor is nicotinamide riboside or nicotinamide mononucleotide.

In a related aspect, the invention provides a kit including magnesium metal; at least one acid; and pyrroloquinoline quinone (PQQ) or salt thereof or a NAD+ precursor. In one embodiments, the kit includes both PQQ or salt thereof and the NAD+ precursor. The magnesium metal may include flakes, e.g., −325 mesh flakes. The magnesium metal may be of 200 mesh or smaller. In some embodiments, the amount of magnesium metal is 5-500 mg. The at least one acid may be selected from the group consisting of maleic acid, succinic acid, malic acid, fumaric acid, formic acid, citric acid, ascorbic acid, oxalic acid, and tartaric acid, or a mixture thereof. In certain embodiments, the at least one acid is of 60 mesh or smaller. The amount of the at least one acid may be 30-4000 mg.

The amount of PQQ or salt thereof may be 1-300 mg. The amount of NAD+ precursor may be 15-2500 mg. In certain embodiments, the NAD+ precursor is nicotinamide riboside or nicotinamide mononucleotide. The magnesium may be in a composition. In certain embodiments, this composition includes only one of the PQQ or salt and the NAD+ precursor. The composition including the magnesium may further include a binding agent, e.g., mannitol, xylitol, maltose, dextrose, or lactose. In other embodiments, the composition including the magnesium further includes water-soluble lubricant, e.g., sodium stearyl fumarate or steric acid. In one embodiment, the PQQ or salt thereof and the NAD+ precursor are formulated together in a composition separately from the magnesium. In other embodiments, the PQQ or salt thereof, NAD+ precursor, and magnesium are formulated in separate compositions.

In another aspect, the invention provides a method of increasing energy or increasing mitochondrial function by administering an effective amount of hydrogen rich water and PQQ or salt thereof or a NAD+ precursor to a subject. The hydrogen rich water may be produced from a composition or kit of invention. In certain embodiments, the PQQ or salt thereof and NAD+ precursor are both administered to the subject. The PQQ or salt thereof or NAD+ precursor may be administered from a composition or kit of the invention.

### DETAILED DESCRIPTION OF THE INVENTION

The present invention provides a composition, that may part of a kit, that dissolves in water to produce hydrogen rich water for use in increasing mitochondrial function and energy production. The composition or kit contains magnesium metal, i.e., elemental magnesium, an edible acid and either pyrroloquinoline quinone (PQQ) in any of its forms, a NAD+ precursor such as nicotinamide mononucleotide or nicotinamide riboside or a combination thereof. In water, the magnesium metal and edible acid react to produce magnesium ions and Hz, which dissolves in the water.

PQQ is a potent supplement promoting mitochondrial biogenesis, while NAD+ precursors such as nicotinamide mononucleotide and nicotinamide riboside are raise levels of the NAD+ coenzyme. Molecular hydrogen activates PGC-1A, the master regulator of mitochondrial biogenesis and likely inhibits AGE formations during Amadori rearrangement primarily through balancing of glutathione to glucose ratios as well as scavenging of super oxide radicals through regulation of the Nrf2 pathway. Molecular hydrogen has shown much promise in regard to keeping homeostatic balances of redox and pro inflammatory cytokine production resulting in decreased damage to mitochondria. By simultaneously protecting against damage while increase beneficial responses, healthy mitochondrial and energy function may be returned to individuals in middle and advanced age.

Accordingly, the invention provides compositions, kits, and methods for increasing mitochondrial function and/or energy levels.

Magnesium Metal

Sufficient magnesium is provided to produce sufficient Hz in the volume of water to which it is added. Accordingly, in certain embodiments, sufficient magnesium to produce at least 0.1 mmol of Hz, e.g., at least 0.5 mmol, 1 mmol, 2 mmol, 3 mmol, 5 mmol, or 10 mmol is provided, e.g., in a tablet. Suitable amounts include 2-800 mg, e.g., 5-500 mg, 10-250 mg, or 20-80 mg of magnesium.

The size and shape of the magnesium may be used to control the rate of reaction. Particles may be spherical, spheroidal, granular, or flaked. Smaller particles and particles with higher surface area to volume ratios react more quickly. Mixtures of various sizes may also be employed. Flaked magnesium has a higher surface area to volume ratio than granular magnesium. In certain embodiments, flaked magnesium of −325 mesh may be employed in the composition. Alternatively or in combination, larger sized magnesium or magnesium with a lower surface to volume ratio relative to flaked may be employed. For example, magnesium of −200 mesh may be employed. In other embodiments, magnesium of +100, −100+200, −200 (e.g., −200+325), −325, or smaller. In certain embodiments, the magnesium is supplied in two sizes, e.g., −200 and −325, with the smaller size being 20-50% of the total and the larger size being the balance.

Edible Acid

Any water soluble edible acid may be employed in the invention. Examples of edible acids include maleic acid, succinic acid, malic acid, fumaric acid, formic acid, citric acid, ascorbic acid, oxalic acid, and mixtures thereof. Other acids include tartaric acid, which may be employed in a mixture with other acids described herein. The acid may be present in an amount to react completely with the magnesium metal, e.g., to provide a final pH of less than 7, when the tablet is placed in water. Alternatively, the pH of the water may be basic after the acid is consumed. In certain embodiments, the amount of acid chosen is sufficient to produce a pH of less than 6, e.g., between 4 and 6. In certain embodiments, the number of moles of acid protons in the acid is at least 10, 20, 30, 40, 50, 75, or 100% greater than the number of moles of magnesium metal present. An exemplary edible acid is malic acid.

Other Components

The invention may also employ PQQ or a salt thereof (e.g., disodium salt) and/or a NAD+ precursor (e.g., nicotinamide riboside or nicotinamide mononucleotide). In certain embodiments, a suitable amount of PQQ or salt thereof is between 1-300 mg, e.g., 5-100 mg, 10-30 mg, or 5-30 mg. In certain embodiments, a suitable amount of NAD+ precursor, e.g., nicotinamide mononucleotide or nicotinamide riboside is between 15-2500 mg, e.g., 20-250 mg, e.g., 40-250 mg or 150-250 mg. In certain embodiments, the invention employs PQQ and nicotinamide riboside.

Additional Components

A composition of the invention may also include additional components, such as a polysaccharide, sweetener, flavoring, coloring, lubricant, or coating. Suitable sweeteners are known in the art, e.g., sucrose, mannose, sucralose, aspartame, saccharin, stevia, and acesulfame K. A composition, e.g., tablet, may also include any food grade coloring and/or flavoring, such as a fruit flavoring. A composition of the invention may also contain a polysaccharide, such as pectin, psyllium fiber, methyl cellulose, various starches, apple powder, lemon powder, lime powder, or grapefruit powder. Polysaccharides may increase the amount of H₂ retained in the liquid after reaction. A composition may further include a water soluble lubricant such as sodium stearyl fumarate. A composition may also have a water penetrable coating to delay the onset on the reaction. For example, a composition may have a coating that dissolves in under 5 minutes, e.g., under 1 minute, to allow the user to close a container before the composition begins to dissolve and H₂ production begins.

Compositions may also include a binding agent. Any binding agent capable of disintegrating in water may be employed, e.g., in Remington (Remington: The Science and Practice of Pharmacy, (22nd ed.) ed. L. V. Allen, Jr., 2013, Pharmaceutical Press, Philadelphia, Pa.). Examples of binding agents include sugars such as maltose, dextrose, and lactose, and sugar alcohols such as mannitol and xylitol. Exemplary binding agents for compositions of the invention include lactose and dextrose. Other binding agents for compositions are known in the art. The amount of binding agent is, for example, between 10 and 50% of the weight of the composition, e.g., between 20-30%. Compositions of the invention may include a single binding agent, such as lactose, or may be made from a combination of two or more binding agents to control the physical properties of the composition.

Forms

A composition of the invention can be manufactured into a number of delivery systems. For example, the composition may be provided in the form of a powder, e.g., a loose powder, powder inside a water soluble capsule or water permeable bag, or small beads, or a film. Another non-limiting example is a composition of the invention combined with a lubricant and binding agent to be pressed into a tablet for oral ingestion. The composition of the invention can be delivered as multiple dosage forms, e.g., each form containing 1, 2, or 3 of the components.

A tablet derived from a composition of the invention may be of any suitable shape. For example, the tablet may be a disk, a sphere, or an ovoid. A single tablet will typically include the amount of magnesium and edible acid required to produce the desired amount of H₂ in a given volume of water, e.g., 500 mL. However, a combination of multiple, smaller tablets may be employed. For example, tablets may be sized to provide sufficient H₂ in 250 mL and multiple tablets may be employed for larger volumes. As the reaction of magnesium metal and acid is activated by water, these compositions of the invention will typically be stored in water resistant packaging, such as foil or plastic. The components of the tablet will typically also be non-hygroscopic, but hygroscopic ingredients may be employed if the tablet is packaged dry in a waterproof container, e.g. blister pack or wrapper. Tablets may be formed by methods known in the art. When the composition is pressed into a tablet, any edible binding agent capable of disintegrating in water may be employed. Examples of binding agents include mannitol, xylitol, maltose, and lactose. Other binding agent for tablets are known in the art. The amount of binding agent is, for example, between 10 and 50% of the weight of the tablet, e.g., between 20-30%.

Additional components such as PQQ or salt thereof and/or NAD+ precursor may be included the same composition with magnesium or one or more additional compositions, e.g., power, liquid, or tablet. Thus, the invention may feature kits of a composition including magnesium and one or more compositions include PQQ or a salt thereof and/or a NAD+ precursor. For example, the kit may feature one or more dosage forms, e.g., tablets, including magnesium and one or more dosage forms including PQQ or salt thereof, NAD+ precursor, or PQQ or salt thereof and NAD+ precursor. When both are present, PQQ or salt thereof and NAD+ precursor may be formulated in the same composition or separate composition or one may be formulated with the magnesium while the other is formulated separately.

Additional compositions including magnesium that may be employed in the present invention are described in WO 2018/011634, which is hereby incorporated by reference.

Containers

Various containers may be used to contact a composition of the invention with a volume of water. In one embodiment, the container has a lid that can be used to seal the container, e.g., shortly after introducing the composition into a volume of water. A sealed container retains H₂ produced while the reaction proceeds to completion. An example of a suitable container is a double walled, double gasketed stainless steel bottle.

Methods of Use

Compositions of the invention including magnesium are used by contacting them with water. The water may also contain other ingredients, e.g., it can be or contain fruit juice. Typically, the amount of water is between 100 mL and 2 L, e.g., 250 mL, 355 mL, 500 mL, 750 mL, or 1 L. The user can add the composition to the water in a sealable container and allow the reaction to proceed for 1 or more minutes, e.g., at least 5 min, 10 min, 15 min, 30 min, 45 min, 60 min, 90 min, or 12 h. In general, the longer the reaction is allowed to proceed, the greater the concentration of H₂ in the water. Preferably, the composition and volume of water produce a concentration of at least 0.5 mM, e.g., at least 1 mM, at least 3 mM, at least 5 mM, or at least 10 mM, e.g., between 5-10 mM. As discussed above, the inclusion of a polysaccharide, either in the composition, or in the water, e.g., in fruit juice, may increase the concentration of H₂ relative to the reaction in the absence of the polysaccharide, either locally near the polysaccharide or in the liquid as a whole.

Additional components, e.g., pyrroloquinoline quinone or a salt thereof and/or a NAD+ precursor may be present in the composition with magnesium or in one or more separate compositions. When administered, the additional components are consumed within 30 minutes prior to 30 minutes after consumption of hydrogen rich water formed from the composition including magnesium. In certain embodiments, additional components are consumed within 15 minutes prior to 15 minutes after consumption of hydrogen rich water, e.g., within 10 minutes prior to 10 minutes after or within 5 minutes prior to 5 minutes after. For example, all components may be consumed simultaneously either by including all components in a single composition or by adding additional components to the water to which the magnesium composition is added. In other

Consumption of hydrogen rich water aids in increasing mitochondrial function by stimulating energy metabolism (Ohta, Pharmacol. Therapeut. 2014, 144, 1-11) and pyrroloquinoline quinone ora salt thereof, and optionally a NAD+ precursor, such as nicotinamide riboside or nicotinamide mononucleotide, synergistically increase this effect.

The methods of the invention may be employed on any appropriate subject. In one embodiment, the subject is at least 18 years old, e.g., at least 25, 35, 45, 55, 65, or 75 years old.

### Example

An exemplary composition includes the following components:

20-80 mg magnesium

5-30 mg, e.g., 10-30, pyrroloquinoline quinone

40-250, e.g., 150-250, mg nicotinamide riboside

Results of Testing

A tablet containing 80 mg magnesium with sufficient organic acids, 20 mg PQQ, and 150 mg nicotinamide riboside elicited in a subject a euphoric response, elevated heart rate followed by heightened senses and lack of need for sleep for roughly 48 hours. The subject noted, measured by a personal activity tracker that, within 5 minutes of drinking the beverage, a 30 second period of euphoria and inability to speak occurred, and immediately after heart rate (HR) climbed from a resting HR in the high 50's to over 100, where it remained for a significant period of time. Thereafter HR settled in the 80s, and the subject slept less than 2 hrs the following two evenings without grogginess or typical symptoms of sleep deprivation. The experiment was conducted again with the same subject using 80 mg of nicotinamide mononucleotide instead of 150 mg of nicotinamide riboside, and the subject reported almost identical outcomes. Further, the subject and 9 other volunteers in an unblinded manner tested compositions of 80 mg magnesium with sufficient acids, 5 mg of PQQ and either 30 mg of nicotinamide mononucleotide (half of subjects) or 50 mg of nicotinamide riboside (remaining half of subjects). All 10 subjects reported slightly elevated heart rates with seemingly heightened senses. Although all 10 subjects described the experience as a slightly altered state, none experienced euphoria in these doses, and none reported any difference in sleep the following evening.

Another experiment was conducted with a separate subject in an unblinded manner. Capsules containing nicotinamide riboside and PQQ were dissolved in water, followed by a tablet containing 80 mg of magnesium with sufficient acids to react to create Hz. The capsules contained 20 mg of PQQ and 150 mg of nicotinamide riboside, as stated on the labels. While an elevated state was experienced, no euphoria or extreme heart rate increase was noted. Slight heart rate increases with an increase in perceived senses were reported. Potential reasons for a decrease in efficacy in similar doses could be label statement doses are higher than actual in capsules, excipients used in capsules could interfere with reactions to create Hz or synergy, or bubbles of Hz could capture active ingredients for altered delivery

What is claimed is:

1. A composition comprising:
magnesium metal;
at least one acid; and
pyrroloquinoline quinone (PQQ) or salt thereof or a NAD+ precursor.

2. The composition of claim 1, comprising PQQ or salt thereof and the NAD+ precursor.

3. The composition of claim 1, in the form of a tablet.

4. The composition of claim 1, wherein the magnesium metal comprises flakes.

5. The composition of claim 1, wherein the magnesium metal comprises −325 mesh flakes.

6. The composition of claim 1, wherein the magnesium metal is of 200 mesh or smaller.

7. The composition of claim 1, wherein the amount of magnesium metal is 5-500 mg.

8. The composition of claim 1, wherein the at least one acid is selected from the group consisting of maleic acid, succinic acid, malic acid, fumaric acid, formic acid, citric acid, ascorbic acid, oxalic acid, and tartaric acid, or a mixture thereof.

9. The composition of claim 1, wherein the at least one acid is of 60 mesh or smaller.

10. The composition of claim 1, wherein the amount of the at least one acid is 30-4000 mg.

11. The composition of claim 1, further comprising a binding agent.

12. The composition of claim 11, wherein the binding agent is mannitol, xylitol, maltose, dextrose, or lactose.

13. The composition of claim 1, further comprising a water-soluble lubricant.

14. The composition of claim 13, where in the water-soluble lubricant is sodium stearyl fumarate or steric acid.

15. The composition of claim 1, wherein the amount of PQQ or salt thereof is 1-300 mg.

16. The composition of claim 1, wherein the amount of NAD+ precursor is 15-2500 mg.

17. The composition of claim 1, wherein the NAD+ precursor is nicotinamide riboside or nicotinamide mononucleotide.

18. A kit comprising:
magnesium metal;
at least one acid; and
PQQ or salt thereof or a NAD+ precursor.

19. The kit of claim 18, comprising PQQ or salt thereof and the NAD+ precursor.

20. The kit of claim 18, wherein the magnesium metal comprises flakes.

21. The kit of claim 18, wherein the magnesium metal comprises −325 mesh flakes.

22. The kit of claim 18, wherein the magnesium metal is of 200 mesh or smaller.

23. The kit of claim 18, wherein the amount of magnesium metal is 5-500 mg.

24. The kit of claim 18, wherein the at least one acid is selected from the group consisting of maleic acid, succinic acid, malic acid, fumaric acid, formic acid, citric acid, ascorbic acid, oxalic acid, and tartaric acid, or a mixture thereof.

25. The kit of claim 18, wherein the at least one acid is of 60 mesh or smaller.

26. The kit of claim 18, wherein the amount of the at least one acid is 30-4000 mg.

27. The kit of claim 18, wherein the magnesium is formulated in a composition.

28. The kit of claim 27, wherein the composition further comprises a binding agent.

29. The kit of claim 28, wherein the binding agent is mannitol, xylitol, maltose, dextrose, or lactose.

30. The kit of claim 27, wherein the composition further comprises a water-soluble lubricant.

31. The kit of claim 30, where in the water-soluble lubricant is sodium stearyl fumarate or steric acid.

32. The kit of claim 18, wherein the amount of PQQ or salt thereof is 1-300 mg.

33. The kit of claim 18, wherein the amount of NAD+ precursor is 15-2500 mg.

34. The kit of claim 18, wherein the NAD+ precursor is nicotinamide riboside or nicotinamide mononucleotide.

35. The kit of claim 27, wherein the composition comprises only one of the PQQ or salt and the NAD+ precursor.

36. The kit of claim 19, wherein the PQQ or salt thereof and the NAD+ precursor are formulated together in a composition separately from the magnesium.

37. The kit of claim 19, wherein the PQQ or salt thereof, NAD+ precursor, and magnesium are formulated in separate compositions.

38. A method of increasing energy or increasing mitochondrial function comprising administering an effective amount of hydrogen rich water and PQQ or salt thereof or a NAD+ precursor to a subject.

39. The method of claim 38, wherein the hydrogen rich water is produced from the composition of any one of claims 1-17 or the kit of any one of claims 18-37.

40. The method of claim 38, wherein the PQQ or salt thereof and NAD+ precursor are both administered to the subject.

41. The method of claim 38, wherein the PQQ or salt thereof or NAD+ precursor are administered from the composition of any one of claims 1-17 or the kit of any one of claims 18-37.
